(19) United States
(12) Patent Application Publication
(10) Pub. No.: US 2022/0354753 A1
(43) Pub. Date: Nov. 10, 2022

(54) Hydrogel-Fabric Composite For Cosmetic Applications

(71) Applicant: Alps South, LLC, St. Petersburg, FL (US)

(72) Inventors: Aldo Laghi, Pinellas Park, FL (US); Nathaniel Vint, Palm Harbor, FL (US); David Carson, Clearwater, FL (US)

(21) Appl. No.: 17/739,246

(22) Filed: May 9, 2022

Related U.S. Application Data
(60) Provisional application No. 63/186,381, filed on May 10, 2021.

(51) Int. Cl. A61K 8/02; A61K 8/9789; A61K 8/9767; A61K 8/67; A61K 8/04; A61Q 19/00

(52) U.S. Cl. CPC A61K 8/0212; A61K 8/9789; A61K 8/9767; A61K 8/671; A61K 8/673; A61K 8/676; A61K 8/678; A61K 8/67; A61K 8/042; A61Q 19/00

(57) ABSTRACT

A hydrogel-fabric composite for cosmetic and therapeutic applications. This application allows the shape retaining solidified gel structure or multi gel composite of a desired hydrogel or blend of hydrogels to disperse the active solutes within the gel more evenly while maintaining activity and then resulting in a set gel shape that conforms with an underlying fabric substrate. Specifically, having the active solutes dispersed within the gel provides an even concentration of active solutes and penetrates the skin over a set amount of time due to the concentration gradient. Further, the temperature constraints of active solute ingredients while still suspending them within the hydrogel for cosmetic use allow for adequate suspension and dissolution of active ingredients throughout the final gel product. Shape retaining Hydrogels, when used with specific active solutes, will remain translucent inferring the purity of the product. Preferred hydrogels to be used in cosmetic facemasks include agar, agarose, carrageenan, carbomers, carboxymethyl cellulose, ethyl cellulose, gelatin, hydroxyethyl cellulose, hydroxypropyl cellulose, magnesium aluminum silicate (veegum), methylcellulose, poloxamers, polyvinyl alcohol, sodium alginate, tragacanth gum, and xanthan hum. Use of a specific hydrogel from this list will depend on which active ingredients are desired to be included in the final product. The process described herein allows for increased dissolution of active ingredients into the final product.

### CROSS-REFERENCE TO RELATED APPLICATIONS

This application claims priority to provisional application number 63/186,381, filed May 10, 2021 entitled “Hydrogel-Fabric Composite for Cosmetic Applications,” the disclosure of which is incorporated herein in its entirety.

### FIELD OF THE INVENTION

This disclosure related to as a shape retaining solid hydrogel or blend of semisolid and solid hydrogels for cosmetic and therapeutic applications. Specifically, this disclosure related to a cosmetic mask or patch that uses a hydrophilic base formula that solidifies into a solid gel which allows for the uniform distribution of active ingredient solutes throughout the solidified gel surface or as a multi-gel composite dispersing solutes in a solid gel within a semisolid gel.

### BACKGROUND OF THE INVENTION

Cosmetic face masks are widely accepted in the marketplace as they enhance the efficacy of any cosmetic or therapeutic agent when compared to the corresponding liquid composition. The enhancement is due to the fact that the contact time between the liquid solution of therapeutic agents which is soaked in the fabric of the mask or patch and the skin is increased greatly when compared to an equivalent liquid as opposed to fabric impregnated solution that, applied on the skin, rapidly evaporates.

The fabric of the mask or patch provides the following important functions: (1) it allows a greater amount of liquid to be stored in contact with the skin even on vertical surfaces; (2) it provides a uniform application of the active agents; and (3) it allows a longer time before drying, which, in turn allows a greater amount of active agents to absorbed by the skin. This is because, typically, when a topical composition dries out, it is no longer able to diffuse the agents into the skin as readily.

Face masks, specifically, have gained rapid worldwide acceptance in the cosmetic industry because of the visible difference in efficacy, observed by users, between masks and topical compositions, such as creams or serums. This is notwithstanding the sloppy and messy process required for application.

Currently, masks are soaked with a liquid and the liquid drips on hands, faces, clothes, etc. Additionally, the removal of the masks or patches from their container is also messy and inconvenient due to dripping, as the masks or patches generally incorporate wet ingredients that need to be unfolded and then placed on the skin.

Certain active ingredients are beneficial for skin health. Skin health is detrimentally affected by free radicals, for example. Human bodies naturally produce free radicals but they also come from exposure to environmental factors like ultraviolet radiation, pollution, cigarette smoke, and other chemicals. Too many free radicals causes oxidative stress to the human system which is an imbalance between the production of free radicals and the ability of the body to counteract or detoxify their harmful effects. Studies have shown that antioxidants in a skin care regimen can help file free radical damage to the skin and help it recover from oxidative stress.

Many vitamins are also helpful to skin health. Vitamin A belongs to a large class of compounds called retinoids. It's precursor is called beta-carotene and is found in cosmetics as retinol, retinyl esters, retinoic acid, retinyl palmitate, acitretin and adapalene. Vitamin A has a variety of effects in the body such as decreasing inflammation, helping the immune system and regulating growth of epidermal cells. Vitamin A has been shown to reverse photoaging and reduce the appearance of wrinkles and also works as a treatment for acne while also potentially having skin lightening effects.

Vitamins E, C, B, and D are likewise good for skin health. Vitamin E soaks up free radicals and has shown the effects of skin smoothening, moisturizing, prevention of pre-mature skin aging, and suppression of UV-inducing erythema when applied topically. Vitamin C, the biologically active form of ascorbic acid, is a potent antioxidant that neutralizes free radicals. Vitamin B is a coenzyme of Vitamin A and helps in cell metabolism. Finally, Vitamin D is a steroid-derived hormone that impacts calcium levels in the body and is produced by the body when exposed to ultraviolet light.

Whitening agents are also beneficial for skin health in individuals with certain conditions. These agents intervene in the melanogenesis process by inhibiting tyrosinase induction and dispersing the pigments in the keratinocytes while also accelerating epidermal turnover. Likewise, moisturizing agents such as humectants, occlusivity agents, keratolytic agents, emollients, and chelating agents.

Therefore, it is an object of this invention to provide an improvement which over comes the inadequacies of the prior art and provides an improvement which is significant to the advancement of the cosmetic application art.

A further object of the invention is to provide a method to enable temperature sensitive compounds to be utilized in a hydrogel matrix that is liquid at mixing temperature but solid and in a fixed shape at room temperature.

A further object of the invention is to provide a method for the creation of gel products for use with cosmetics in specific shapes and conformities.

A further object of the invention is to provide a face mask product that incorporates a shape-retaining hydrogel product.

A further object of the invention is to provide a face mask product that does not drip by using a blend of a solid hydrogel and semisolid hydrogel.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a perspective view of the preferred final gel product as a mask;

FIG. 2 is a front view of the preferred final gel product as a mask;

FIG. 3 is a cross-sectional view of FIG. 2 along line B-B;

FIG. 4 is a front view of the solidified gel and gel solution mask; and

FIG. 5 is a cross sectional view of FIG. 4.

### SUMMARY OF THE INVENTION

For the purpose of summarizing this invention, this invention comprises a solid hydrogel-fabric composite or semisolid blend of gels and fabric composite for cosmetic and therapeutic applications. This application allows the shape-retaining gel structure of a desired hydrogel to disperse the active solutes within the gel evenly while maintaining activity and then resulting in a set gel shape that conforms with an underlying fabric substrate. Specifically, having the active solutes dispersed within the gel provides an even concentration of active solutes and penetrates the skin over a set amount of time due to the concentration gradient. Further, the temperature constraints of active solute ingredients while still suspending them within the hydrogel for cosmetic use allow for adequate suspension and dissolution of active ingredients throughout the final gel product.

Preferred hydrogels to be used in cosmetic facemasks include agar, agarose, carrageenan, carbomers, carboxymethyl cellulose, ethyl cellulose, gelatin, hydroxyethyl cellulose, hydroxypropyl cellulose, magnesium aluminum silicate (veegum), methylcellulose, poloxamers, polyvinyl alcohol, sodium alginate, tragacanth gum, and xanthan gum. Use of a specific hydrogel from this list will depend on which active ingredients are desired to be included in the final product. The process described herein allows for dissolution of active ingredients into the final product.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims. The various components of the present invention, and the manner in which they interrelate, are described in greater detail hereinafter.

Hydrogel solutions that gel at the higher temperature allow for the adequate suspension of active ingredients within the formed three dimensional structure for cosmetic use. Preferred hydrogels to be used in cosmetic facemasks include agar, agarose, carrageenan, carbomers, carboxymethyl cellulose, ethyl cellulose, gelatin, hydroxyethyl cellulose, hydroxypropyl cellulose, magnesium aluminum silicate (veegum), methylcellulose, poloxamers, polyvinyl alcohol, sodium alginate, tragacanth gum, and xanthan gum. The wide range of thermal hysteresis between liquid to ridged gel transition allows for the suspension of active solutes within the gel and the use of temperature sensitive components that can be suspended later in process before the final application of solution towards a final product.

Preferably, the active ingredients to be used in the hydrogel matrix of the present invention include, but are not limited to:
natural extracts of the following:
   green tea;
   rosemary;
   grape seed;
   basil grape;
   blueberry;
   tomato;
   acerola seed;
   pine bark; and
   milk thistle;
Vitamins A, B, C, D, or E
Whitening agents such as hydroquinione and its derivatives, retinoic acid, tretinoin, or tyrosinase inhibitors;
Humectants such as glycerin, propylene glycol, butylene glycol, urea, glucosamine, or lanolin;
Occlusivity agents such as petrolatum, isododecane, C30-45 alkyl methicone, or shea or other nut butters;
Keratolytic agents such as salicylic acids and its derivatives, alpha hydroxyl acid, or allantoin;
Emollients such as dimethicone gum, rosspolymer, amodimethicone, cetyl palmitate, caprylic triglyceride, dicapryl ether, isoeicosane, olive oil, or related esters/transesters;
Chelating agents such as trisodium EDTA; and
Other active ingredients that do not degrade at temperatures higher than 70 c can also be used.

These active ingredients are chosen specifically because they do not lose their activity when mixed with the preferred hydrogel materials at a temperature between 45 and 90 degrees Celsius while at the same time diffusing out of the gel and transferring to the skin when applied to the user's skin at room temperature.

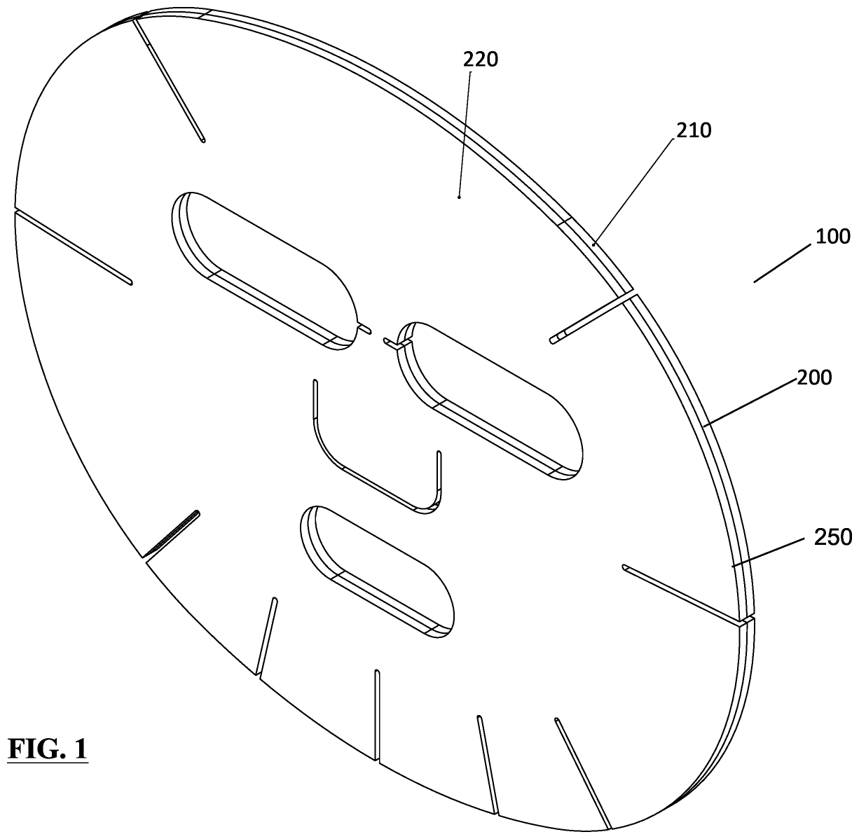

As shown in FIG. 1, the hydrogel cosmetic application, 100, can be shaped into any moldable shape but are most preferably shaped as a mask. The hydrogel cosmetic application can also be made into patches. The hydrogel cosmetic application comprises a fabric substrate which can be woven, non-woven, or knit and made of materials such as cotton, bamboo, cellulose, polypropylene, polyester, polyethylene, polyamide, acrylic and vinyl. Choice of construction and material is dependent on degree of absorption needed for the hydrogel, the mechanical characteristics of the substrate, and cost. The solidified hydrogel containing active ingredients is of the same shape as the shape of a fabric substrate because the liquid gel impregnates the fabric substrate at a temperature between 45 and 90 degrees Celsius and then solidifies at room temperature retaining the shape of the fabric substrate. Alternatively, the liquid hydrogel can be applied to only a single side of the fabric substrate such that only half of the volume of the fabric substrate is impregnated with hydrogel along a latitudinal axis. Further, these final gel products preferably have a thickness around 1.0 millimeters but can be any thickness between 0.3 and 3 millimeters dependent on user preference.

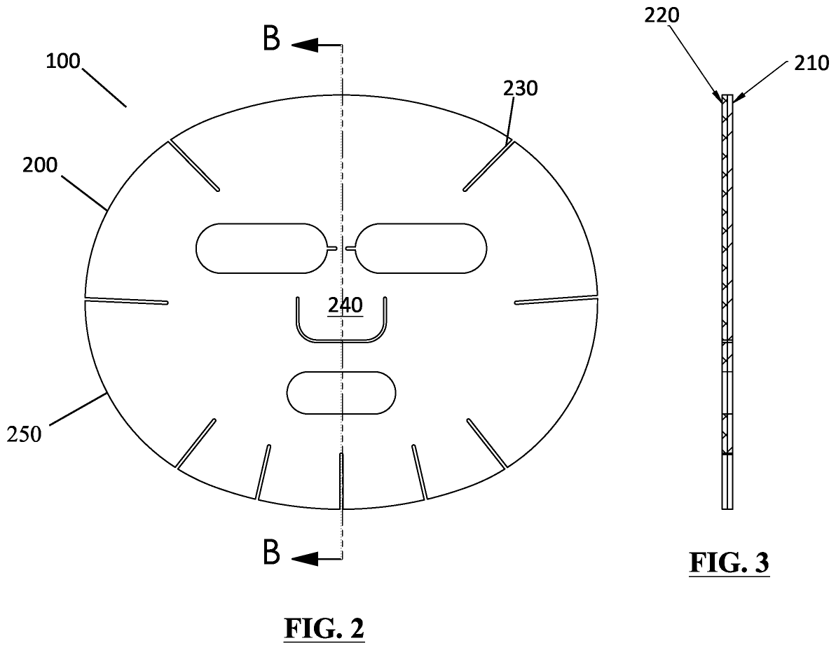

As seen in FIG. 2 and FIG. 3, the hydrogel cosmetic application 100 can be in the form of a mask 200 comprising two layers, a fabric substrate 210 and the hydrogel layer 220. The mask version of the hydrogel cosmetic application 100 preferably has a plurality of slits 230 projecting towards a center point 240 of the mask 200 from an outer edge 250. When the hydrogel layer 220 dries at room temperature, the hydrogel layer 220 can be visible to the user and distinct from the fabric substrate 210. Alternatively, after the hydrogel layer 220 dries at room temperature, the hydrogel layer 220 may form a unitary layer with the fabric substrate 210 dependent on the hydrogel that forms the basis of the hydrogel layer 220.

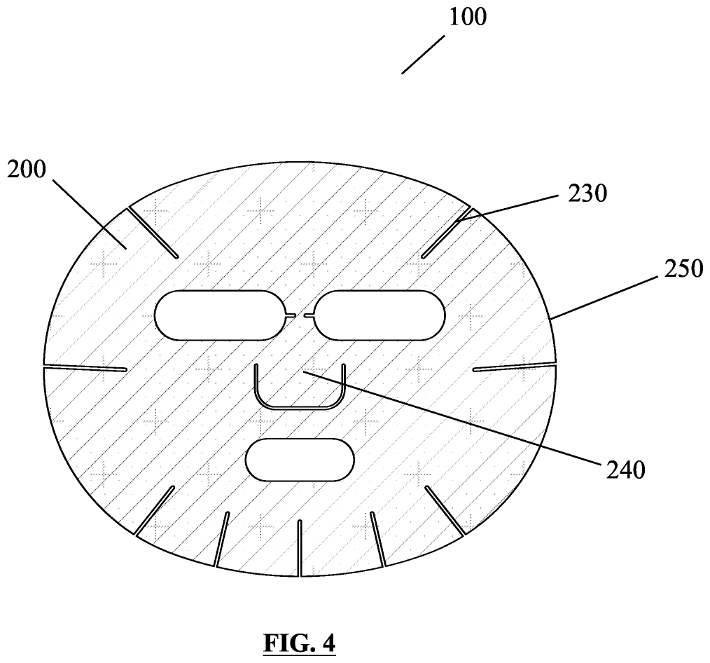

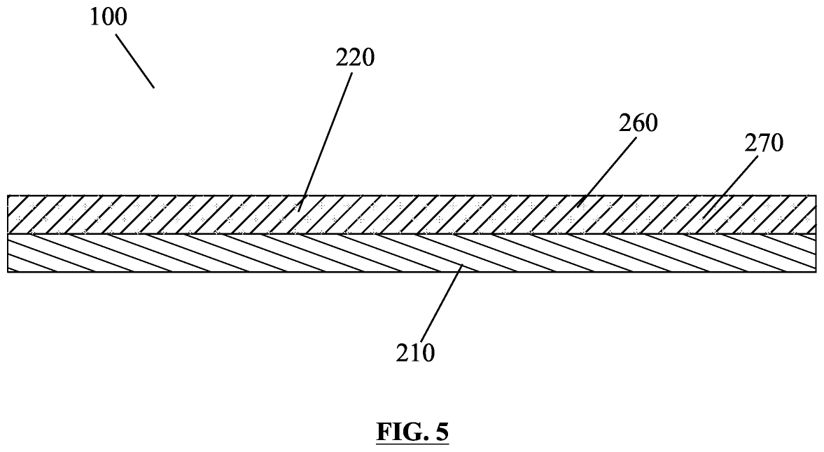

As shown in FIG. 4 and FIG. 5, a second embodiment of the invention is provided by applying multiple hydrogel agents. A semisolid gel 260 with a secondary solid hydrogel 270 within the semisolid gel 260 to create the hydrogel layer 220 allows impregnation of folding masks while removing the dripping nature of common use folding masks. This second embodiment also preserves the advanced characteristics of a solidified gel product and the solute concentration it contains.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A hydrogel cosmetic application comprising:
a fabric substrate; and
a hydrogel layer impregnated into said fabric substrate forming a composite wherein said hydrogel layer further comprises at least one suspended active ingredient wherein said active ingredient does not degrade at temperatures higher than 70 degrees Celsius.

2. The hydrogel cosmetic application of claim 1 wherein the active ingredient is chosen from the group consisting of natural extract of green tea, natural extract of rosemary, natural extract of grape seed, natural extract of basil grape, natural extract of blueberry, natural extract of tomato, natural extract of acerola seed, natural extract of pine bark, natural extract of milk thistle, vitamin A, vitamin B, vitamin C, Vitamin D, Vitamin E, a whitening agent, a humectant, a occlusivity agent, a keratolytic agent, an emollient, and a chelating agent.

3. The hydrogel cosmetic application of claim 1 wherein the hydrogel cosmetic application is formed into the shape of a facemask.

4. The hydrogel cosmetic application of claim 3 wherein the facemask comprises a plurality of slits projecting towards a center point of the facemask from an outer edge.

5. The hydrogel cosmetic application of claim 1 wherein the hydrogel layer is capable of visual perception to a user.

6. The hydrogel cosmetic application of claim 1 wherein the hydrogel layer is incapable of visual perception to a user.

7. The hydrogel cosmetic application of claim 1 wherein the hydrogel cosmetic application has a thickness between 0.3 and 3 millimeters.

8. A hydrogel cosmetic application comprising:
a fabric substrate;
a gel layer comprising a mixture of a semisolid hydrogel when at room temperature, a solid hydrogel when at room temperature, and at least one active ingredient that does not degrade at temperatures higher than 70 degrees Celsius, wherein said gel layer is impregnated into said fabric substrate forming a composite.

9. The hydrogel cosmetic application of claim 8 wherein the active ingredient is chosen from the group consisting of natural extract of green tea, natural extract of rosemary, natural extract of grape seed, natural extract of basil grape, natural extract of blueberry, natural extract of tomato, natural extract of acerola seed, natural extract of pine bark, natural extract of milk thistle, vitamin A, vitamin B, vitamin C, Vitamin D, Vitamin E, a whitening agent, a humectant, a occlusivity agent, a keratolytic agent, an emollient, and a chelating agent.

10. The hydrogel cosmetic application of claim 8 wherein the hydrogel cosmetic application is formed into a facemask shape.

11. The hydrogel cosmetic application of claim 10 wherein the facemask comprises a plurality of slits projecting towards a center point of the facemask from an outer edge.

12. The hydrogel cosmetic application of claim 8 wherein the hydrogel layer is capable of being visually perceived by a user.

13. The hydrogel cosmetic application of claim 8 wherein the hydrogel layer is incapable of being visually perceived by a user.

14. The hydrogel cosmetic application of claim 8 wherein the hydrogel cosmetic application has a thickness between 0.3 and 3 millimeters.

15. A hydrogel cosmetic application comprising:
a fabric substrate shaped into a facemask;
a gel layer comprising a blend of a semisolid hydrogel when at room temperature, a solid hydrogel when at room temperature, and at least one active ingredient that does not degrade at temperatures higher than 70 degrees Celsius, wherein said gel layer is impregnated into said fabric substrate forming a composite; and
wherein said active ingredient is chosen from the group consisting of natural extract of green tea, natural extract of rosemary, natural extract of grape seed, natural extract of basil grape, natural extract of blueberry, natural extract of tomato, natural extract of acerola seed, natural extract of pine bark, natural extract of milk thistle, vitamin A, vitamin B, vitamin C, Vitamin D, Vitamin E, a whitening agent, a humectant, a occlusivity agent, a keratolytic agent, an emollient, and a chelating agent.
